(19) United States
(12) Patent Application Publication
(10) Pub. No.: US 2025/0082423 A1
(43) Pub. Date: Mar. 13, 2025

(54) SEMI-AUTOMATIC POSITIONING OF MULTIPLE PASSIVE JOINTS IN A ROBOTIC SYSTEM

(71) Applicant: Covidien LP, Mansfield, MA (US)

(72) Inventors: Tom Baker, Germering (DE); Andreas Tobergte, Wessling (DE); Ulrich Hagn, Muenchen (DE)

(21) Appl. No.: 18/580,256

(22) PCT Filed: Aug. 22, 2022
(86) PCT No.: PCT/US2022/040987

§ 371 Date: Jan. 18, 2024

Related U.S. Application Data
(60) Provisional application No. 63/237,552, filed on Aug. 27, 2021.

(51) Int. Cl. A61B 34/37; A61B 34/00; A61B 34/30

(52) U.S. Cl. CPC A61B 34/37; A61B 34/25; A61B 2034/306

(57) ABSTRACT

A surgical robotic system includes a robotic arm and a setup arm, each having multiple joints. The arms may be set into a passive mode allowing for manual movement of each joint until an aligned configuration is achieved. Once each joint in a desired position, that joint is locked. An aligned configuration is achieved once all of the joints are in the desired position and are locked.

### CROSS-REFERENCE TO RELATED APPLICATION

The present application claims the benefit of and priority to U.S. Provisional Application No. 63/237,552, filed on Aug. 27, 2021, the entire disclosure of which is incorporated by reference herein in its entirety.

### BACKGROUND

### Technical Field

The present disclosure generally relates to surgical robotic systems and in particular to robotic arms having a plurality of passive joints. The disclosure provides for a system and method for assisted positioning of passive joints to configure the robotic arm into a desired configuration.

### Background of Related Art

Surgical robotic systems are currently being used in variety of medical procedures, including minimally invasive medical procedures. Some surgical robotic systems include a surgeon console controlling a surgical robotic arm and a surgical instrument having an end effector (e.g., forceps or grasping instrument) coupled to and actuated by the robotic arm. In operation, the robotic arm is moved to a position over a patient and then guides the surgical instrument into a small incision via a surgical port or a natural orifice of a patient to position the end effector at a work site within the patient's body.

Thus, there is a need for positioning robotic arms having multiple passive joints at the same in order to prepare the surgical robotic system for surgery.

### SUMMARY

In robotic systems, joints enable motion between two rigid bodies (e.g., links). The combined motion of multiple joints allows the robotic system, and in particular, robotic arms to fulfill a specific function. Joints may be active or passive. Active joints may contain a friction brake to stop and hinder any further motion of the robotic arms in certain situations, e.g., in case of an emergency. Active joints may also include an actuator (e.g., a motor) in addition to the friction brake, which enables these joints to achieve a position automatically. Passive robotic joints utilize direct user interaction to achieve re-positioning. As used herein, passive robotic joints also include passively behaving joints, such as an active joint having an actuator that simulates passive behavior, e.g., allowing for back driving of the actuator. This direct user interaction may also be used for risk mitigation, to ensure the user maintains control of the relevant joints.

Accurately positioning multiple passive joints of a robotic system at the same time can be a non-trivial task if there is no separate actuation of each individual joint. Possible situations where this occurs include: startup of the system; transitioning the robotic system out of a storage position; re-positioning joints for specific tasks during nominal operation; and at shutdown to transition the robotic system into a storage position.

The present disclosure simplifies these tasks and provides a semi-automatic positioning mode. When the presently disclosed positioning mode is first entered, the user can freely move all relevant joints. As soon as the user moves a joint to its aligned or desired position, the joint automatically transitions to its non-actuatable state (e.g., by engaging the brake). As a result, if the user has individually moved all joints to their aligned positions, the robotic system as a whole is in the aligned configuration. This mode is extended by conditionally transitioning joints to their non-actuatable state by cross dependencies of other joints. Serial chain robotic systems, such as the robotic arms according to the present disclosure, allow for this action with the joint closest to the base of the robotic system first (e.g., setup arm), before continuing with the next joint in the serial chain.

According to one aspect of the present disclosure, a surgical robotic system is disclosed. The system includes a robotic arm having a plurality of robotic arm joints. The system also includes a controller configured to receive an alignment for the robotic arm including an aligned position for each robotic arm joint of the plurality of robotic arm joints. The controller is also configured to switch each robotic arm joint of the plurality of robotic arm joints into a passive mode during which each robotic arm joint is manually movable and track a position of each robotic arm joint of the plurality of robotic arm joints while each robotic arm joint is manually moved into alignment. The controller is further configured to lock each robotic arm joint of the plurality of robotic arm joints once the aligned position for each robotic arm joint is achieved.

Implementations of the above embodiment may include one or more of the following features. According to one aspect of the above embodiment, the surgical robotic system may further include: a display configured to output an indication that each robotic arm joint of the plurality of robotic arm joints is moved into alignment. The display may be further configured to output an indication that the robotic arm is in an aligned configuration. The display may be configured to output instructions for aligning the robotic arm. The instructions may include aligning the plurality of robotic arm joints in a predetermined sequence. An alignment may correspond to a configuration of the robotic arm for a storage configuration, transport configuration, or an operation configuration. The surgical robotic system may further include an instrument configured to mechanically engage the robotic arm. The surgical robotic system may further include a movable cart having a setup arm configured to engage the robotic arm, the setup arm may include a plurality of setup arm joints. The controller may be further configured to receive an alignment for the setup arm may include an aligned position for each setup arm joint of the plurality of setup arm joints; switch each setup arm joint of the plurality of setup arm joints into a passive mode during which each setup arm joint is manually movable; track a position of each setup arm joint of the plurality of setup arm joints while each setup arm joint is manually moved into alignment; and lock each setup arm joint of the plurality of setup arm joints once the aligned position for each setup arm joint is achieved. The plurality of robotic arm joints and the plurality of setup arm joints may be aligned together.

According to another aspect of the present disclosure, a method for controlling a surgical robotic system is disclosed. The method may include loading an alignment for a robotic arm having a plurality of robotic arm joints. The alignment may include an aligned position for each robotic arm joint of the plurality of robotic arm joints. The method may also include switching each robotic arm joint of the plurality of robotic arm joints into a passive mode and tracking a position of each robotic arm joint of the plurality of robotic arm joints while each robotic arm joint is moved into alignment. The method may also include moving each robotic arm joint of the plurality of robotic arm joints while in the passive mode. The method may further include locking each robotic arm joint of the plurality of robotic arm joints once the aligned position for each robotic arm joint is achieved.

Implementations of the above embodiment may include one or more of the following features. According to one aspect of the above embodiment, the method may further include outputting on a display an indication that each robotic arm joint of the plurality of robotic arm joints is moved into alignment. The method may also include outputting on a display an indication that the robotic arm is in an aligned configuration. The method may include outputting instructions for aligning the robotic arm on a display. Instructions may include aligning the plurality of robotic arm joints in a predetermined sequence. An alignment may correspond to a configuration of the robotic for a storage configuration, transport configuration, or an operation configuration. The method may further include mechanically engaging an instrument to the robotic arm. The method may further include coupling the robotic arm to a movable cart that may include a setup arm configured to engage the robotic arm, and the setup arm may include a plurality of setup arm joints. The method may include: loading an alignment for the setup arm, where the alignment may include an aligned position for each setup arm joint of the plurality of setup arm joints; switching each setup arm joint of the plurality of setup arm joints into a passive mode during which each setup arm joint is manually movable; tracking a position of each setup arm joint of the plurality of setup arm joints while each setup arm joint is manually moved into alignment; and locking each setup arm joint of the plurality of setup arm joints once the aligned position for each setup arm joint is achieved. The method may further include aligning the plurality of robotic arm joints and the plurality of setup arm joints together.

### BRIEF DESCRIPTION OF THE DRAWINGS

Various aspects of the present disclosure are described herein with reference to the drawings wherein:

FIG. 1 is a schematic illustration of a surgical robotic system including a control tower, a console, and one or more surgical robotic arms each disposed on a movable cart according to an aspect of the present disclosure;

FIG. 2 is a perspective view of a surgical robotic arm of the surgical robotic system of FIG. 1 according to an aspect of the present disclosure;

FIG. 3 is a perspective view of a setup arm with the surgical robotic arm of the surgical robotic system of FIG. 1 according to an aspect of the present disclosure;

FIG. 4 is a schematic diagram of a computer architecture of the surgical robotic system of FIG. 1 according to an aspect of the present disclosure;

FIG. 5 is a plan schematic view of movable carts of FIG. 1 positioned about a surgical table according to an aspect of the present disclosure;

FIG. 6 is side view of a surgical robotic arm of FIG. 1 in a first, unaligned, configuration and a second, aligned, configuration; and

FIG. 7 is a flow chart of a method for aligning the surgical robotic arm of FIG. 1 according to an embodiment of the present disclosure.

### DETAILED DESCRIPTION

The term “application” may include a computer program designed to perform functions, tasks, or activities for the benefit of a user. Application may refer to, for example, software running locally or remotely, as a standalone program or in a web browser, or other software which would be understood by one skilled in the art to be an application. An application may run on a controller, or on a user device, including, for example, a mobile device, a personal computer, or a server system.

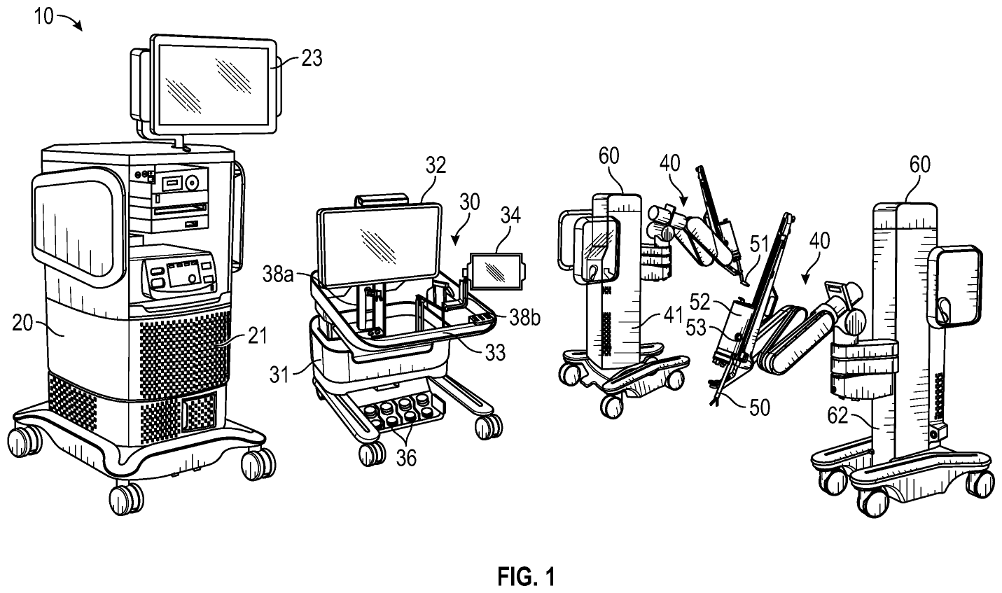

With reference to FIG. 1, a surgical robotic system 10 includes a control tower 20, which is connected to all of the components of the surgical robotic system 10 including a surgeon console 30 and one or more robotic arms 40. Each of the robotic arms 40 includes a surgical instrument 50 removably coupled thereto. Each of the robotic arms 40 is also coupled to a movable cart 60.

The surgical instrument 50 is configured for use during minimally invasive surgical procedures. In aspects, the surgical instrument 50 may be configured for open surgical procedures. In further aspects, the surgical instrument 50 may be an electrosurgical forceps configured to seal tissue by compressing tissue between jaw members and applying electrosurgical current thereto. In yet further aspects, the surgical instrument 50 may be a surgical stapler including a pair of jaws configured to grasp and clamp tissue while deploying a plurality of tissue fasteners, e.g., staples, and cutting stapled tissue.

One of the robotic arms 40 may include an endoscopic camera 51 configured to capture video of the surgical site. The endoscopic camera 51 may be a stereoscopic endoscope configured to capture two side-by-side (i.e., left and right) images of the surgical site to produce a video stream of the surgical scene. The endoscopic camera 51 is coupled to a video processing device 56, which may be disposed within the control tower 20. The video processing device 56 may be any computing device as described below configured to receive the video feed from the endoscopic camera 51 perform the image processing based on the depth estimating algorithms of the present disclosure and output the processed video stream.

The surgeon console 30 includes a first display 32, which displays a video feed of the surgical site provided by camera 51 of the surgical instrument 50 disposed on the robotic arms 40, and a second display 34, which displays a user interface for controlling the surgical robotic system 10. The first and second displays 32 and 34 are touchscreens allowing for displaying various graphical user inputs. The video processing device 56 is configured to process the video feed from the endoscopic camera 51 and to output a processed video stream on the first displays 32 of the surgeon console 30 and/or the display 23 of the control tower 20.

The surgeon console 30 also includes a plurality of user interface devices, such as foot pedals 36 and a pair of handle controllers 38a and 38b which are used by a user to remotely control robotic arms 40. The surgeon console further includes an armrest 33 used to support clinician's arms while operating the handle controllers 38a and 38b.

The control tower 20 includes a display 23, which may be a touchscreen, and outputs on the graphical user interfaces (GUIs). The control tower 20 also acts as an interface between the surgeon console 30 and one or more robotic arms 40. In particular, the control tower 20 is configured to control the robotic arms 40, such as to move the robotic arms 40 and the corresponding surgical instrument 50, based on a set of programmable instructions and/or input commands from the surgeon console 30, in such a way that robotic arms 40 and the surgical instrument 50 execute a desired movement sequence in response to input from the foot pedals 36 and the handle controllers 38a and 38b.

Each of the control tower 20, the surgeon console 30, and the robotic arm 40 includes a respective computer 21, 31, 41. The computers 21, 31, 41 are interconnected to each other using any suitable communication network based on wired or wireless communication protocols. The term “network,” whether plural or singular, as used herein, denotes a data network, including, but not limited to, the Internet, Intranet, a wide area network, or a local area networks, and without limitation as to the full scope of the definition of communication networks as encompassed by the present disclosure. Suitable protocols include, but are not limited to, transmission control protocol/internet protocol (TCP/IP), datagram protocol/internet protocol (UDP/IP), and/or datagram congestion control protocol (DCCP). Wireless communication may be achieved via one or more wireless configurations, e.g., radio frequency, optical, Wi-Fi, Bluetooth (an open wireless protocol for exchanging data over short distances, using short length radio waves, from fixed and mobile devices, creating personal area networks (PANs), ZigBee® (a specification for a suite of high level communication protocols using small, low-power digital radios based on the IEEE 122.15.4-2003 standard for wireless personal area networks (WPANs)).

The computers 21, 31, 41 may include any suitable processor (not shown) operably connected to a memory (not shown), which may include one or more of volatile, non-volatile, magnetic, optical, or electrical media, such as read-only memory (ROM), random access memory (RAM), electrically-erasable programmable ROM (EEPROM), non-volatile RAM (NVRAM), or flash memory. The processor may be any suitable processor (e.g., control circuit) adapted to perform the operations, calculations, and/or set of instructions described in the present disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted for by using any logic processor (e.g., control circuit) adapted to execute algorithms, calculations, and/or set of instructions described herein.

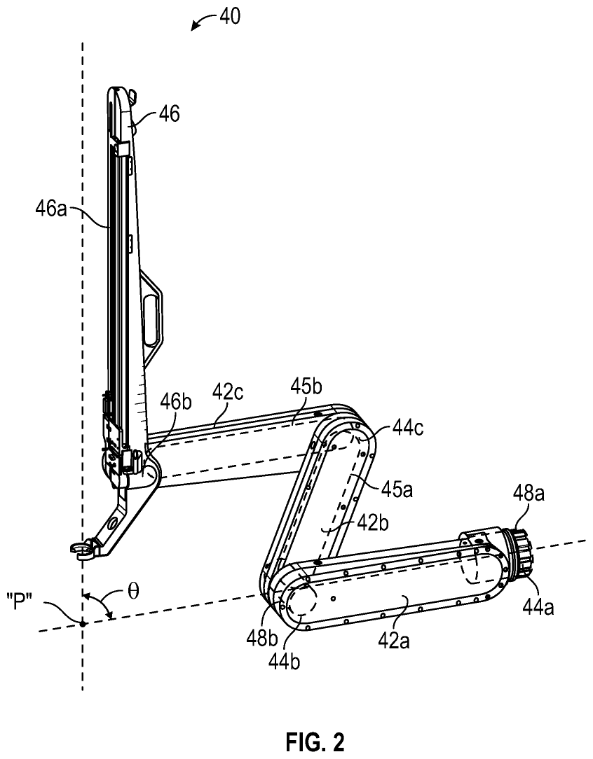

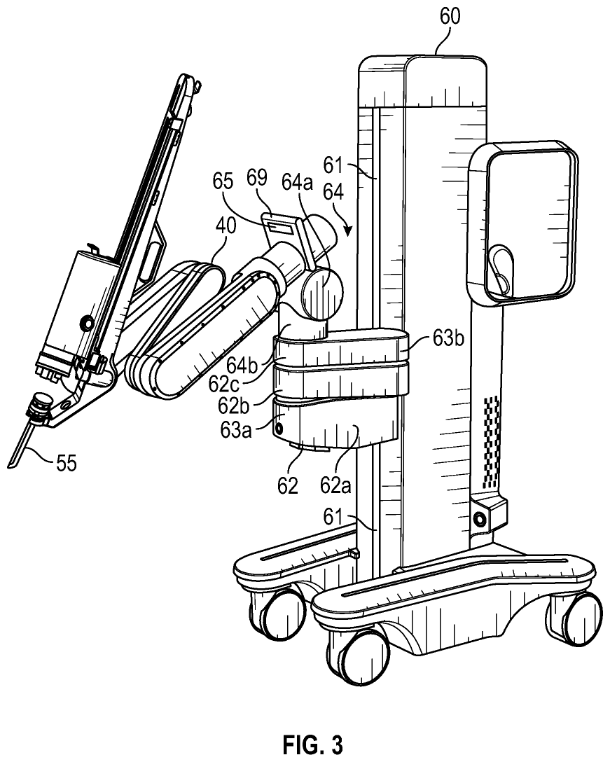

With reference to FIG. 2, each of the robotic arms 40 may include a plurality of links 42a, 42b, 42c, which are interconnected at joints 44a, 44b, 44c, respectively. The joint 44a is configured to secure the robotic arm 40 to the movable cart 60 and defines a first longitudinal axis. With reference to FIG. 3, the movable cart 60 includes a lift 61 and a setup arm 62, which provides a base for mounting of the robotic arm 40. The lift 61 allows for vertical movement of the setup arm 62. The movable cart 60 also includes a display 69 for displaying information pertaining to the robotic arm 40.

The setup arm 62 includes a first link 62a, a second link 62b, and a third link 62c, which provide for lateral maneuverability of the robotic arm 40. The links 62a, 62b, 62c are interconnected at joints 63a and 63b, each of which may include an actuator (not shown) for rotating the links 62b and 62b relative to each other and the link 62c. In particular, the links 62a, 62b, 62c are movable in their corresponding lateral planes that are parallel to each other, thereby allowing for extension of the robotic arm 40 relative to the patient (e.g., surgical table). In aspects, the robotic arm 40 may be coupled to a surgical table. The setup arm 62 includes controls 65 for adjusting movement of the links 62a, 62b, 62c as well as the lift 61.

The third link 62c includes a rotatable base 64 having two degrees of freedom. In particular, the rotatable base 64 includes a first actuator 64a and a second actuator 64b. The first actuator 64a is rotatable about a first stationary arm axis which is perpendicular to a plane defined by the third link 62c and the second actuator 64b is rotatable about a second stationary arm axis which is transverse to the first stationary arm axis. The first and second actuators 64a and 64b allow for full three-dimensional orientation of the robotic arm 40.

The actuator 48b of the joint 44b is coupled to the joint 44c via the belt 45a, and the joint 44c is in turn coupled to the joint 46c via the belt 45b. Joint 44c may include a transfer case coupling the belts 45a and 45b, such that the actuator 48b is configured to rotate each of the links 42b, 42c and the holder 46 relative to each other. More specifically, links 42b, 42c, and the holder 46 are passively coupled to the actuator 48b which enforces rotation about a pivot point “P” which lies at an intersection of the first axis defined by the link 42a and the second axis defined by the holder 46. Thus, the actuator 48b controls the angle θ between the first and second axes allowing for orientation of the surgical instrument 50. Due to the interlinking of the links 42a, 42b, 42c, and the holder 46 via the belts 45a and 45b, the angles between the links 42a, 42b, 42c, and the holder 46 are also adjusted in order to achieve the desired angle θ. In aspects, some or all of the joints 44a, 44b, 44c may include an actuator to obviate the need for mechanical linkages.

The joints 44a and 44b include an actuator 48a and 48b configured to drive the joints 44a, 44b, 44c relative to each other through a series of belts 45a and 45b or other mechanical linkages such as a drive rod, a cable, or a lever and the like. In particular, the actuator 48a is configured to rotate the robotic arm 40 about a longitudinal axis defined by the link 42a. Each of the actuators 48a and b and the actuators of the joints 63a and b may also include a brake to stop rotation of the corresponding joints 44a-c and 64a and b.

With reference to FIG. 2, the robotic arm 40 also includes a holder 46 defining a second longitudinal axis and configured to receive an instrument drive unit (IDU) 52 (FIG. 1). The IDU 52 is configured to couple to an actuation mechanism of the surgical instrument 50 and the camera 51 and is configured to move (e.g., rotate) and actuate the instrument 50 and/or the camera 51. IDU 52 transfers actuation forces from its actuators to the surgical instrument 50 to actuate components (e.g., end effector) of the surgical instrument 50. The holder 46 includes a sliding mechanism 46a, which is configured to move the IDU 52 along the second longitudinal axis defined by the holder 46. The holder 46 also includes a joint 46b, which rotates the holder 46 relative to the link 42c. During endoscopic procedures, the instrument 50 may be inserted through an endoscopic port 55 (FIG. 3) held by the holder 46.

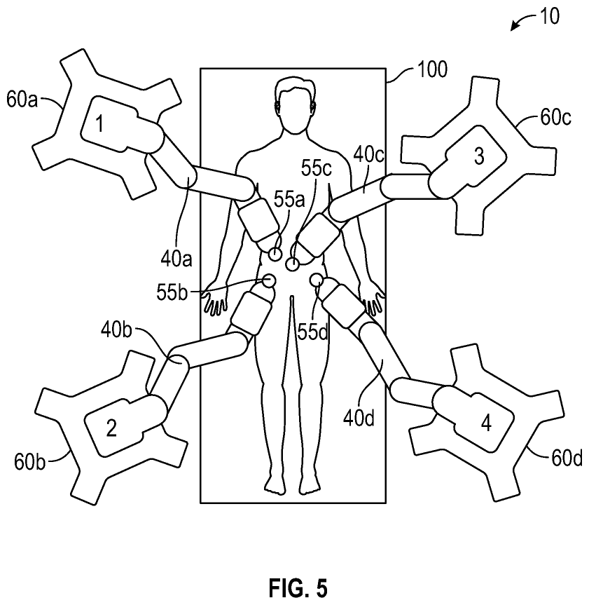

The robotic arm 40 also includes a plurality of manual override buttons 53 (FIGS. 1 and 5) disposed on the IDU 52 and the setup arm 62, which may be used in a manual mode. The user may press one or more of the buttons 53 to move the component associated with the button 53.

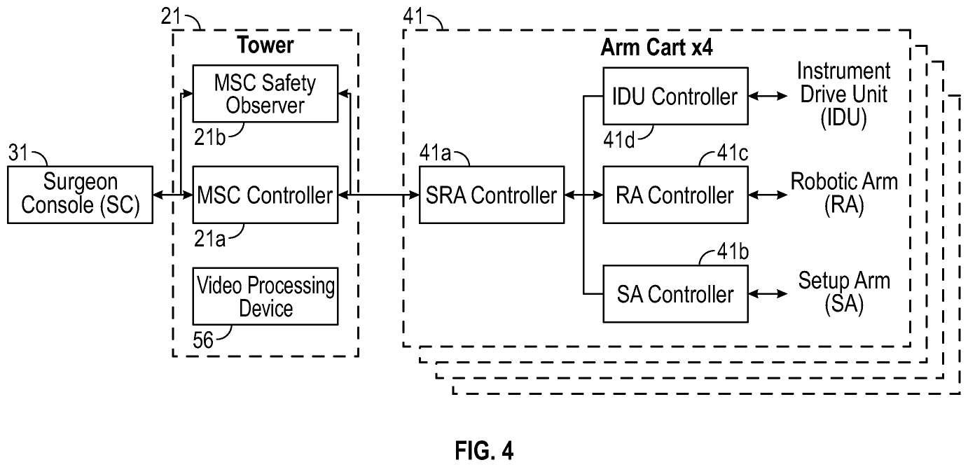

With reference to FIG. 4, each of the computers 21, 31, 41 of the surgical robotic system 10 may include a plurality of controllers, which may be embodied in hardware and/or software. The computer 21 of the control tower 20 includes a controller 21a and safety observer 21b. The controller 21a receives data from the computer 31 of the surgeon console 30 about the current position and/or orientation of the handle controllers 38a and 38b and the state of the foot pedals 36 and other buttons. The controller 21a processes these input positions to determine desired drive commands for each joint of the robotic arm 40 and/or the IDU 52 and communicates these to the computer 41 of the robotic arm 40. The controller 21a also receives the actual joint angles measured by encoders of the actuators 48a and 48b and uses this information to determine force feedback commands that are transmitted back to the computer 31 of the surgeon console 30 to provide haptic feedback through the handle controllers 38a and 38b. The safety observer 21b performs validity checks on the data going into and out of the controller 21a and notifies a system fault handler if errors in the data transmission are detected to place the computer 21 and/or the surgical robotic system 10 into a safe state.

The computer 41 includes a plurality of controllers, namely, a main cart controller 41a, a setup arm controller 41b, a robotic arm controller 41c, and an instrument drive unit (IDU) controller 41d. The main cart controller 41a receives and processes joint commands from the controller 21a of the computer 21 and communicates them to the setup arm controller 41b, the robotic arm controller 41c, and the IDU controller 41d. The main cart controller 41a also manages instrument exchanges and the overall state of the movable cart 60, the robotic arm 40, and the IDU 52. The main cart controller 41a also communicates actual joint angles back to the controller 21a.

The setup arm controller 41b controls each of joints 63a and 63b, and the rotatable base 64 of the setup arm 62 and calculates desired motor movement commands (e.g., motor torque) for the pitch axis and controls the brakes. The robotic arm controller 41c controls each joint 44a and 44b of the robotic arm 40 and calculates desired motor torques required for gravity compensation, friction compensation, and closed loop position control of the robotic arm 40. The robotic arm controller 41c calculates a movement command based on the calculated torque. The calculated motor commands are then communicated to one or more of the actuators 48a and 48b in the robotic arm 40. The actual joint positions are then transmitted by the actuators 48a and 48b back to the robotic arm controller 41c.

The IDU controller 41d receives desired joint angles for the surgical instrument 50, such as wrist and jaw angles, and computes desired currents for the motors in the IDU 52. The IDU controller 41d calculates actual angles based on the motor positions and transmits the actual angles back to the main cart controller 41a.

The robotic arm 40 is controlled in response to a pose of the handle controller controlling the robotic arm 40, e.g., the handle controller 38a, which is transformed into a desired pose of the robotic arm 40 through a hand eye transform function executed by the controller 21a. The hand eye function, as well as other functions described herein, is/are embodied in software executable by the controller 21a or any other suitable controller described herein. The pose of one of the handle controller 38a may be embodied as a coordinate position and role-pitch-yaw (“RPY”) orientation relative to a coordinate reference frame, which is fixed to the surgeon console 30. The desired pose of the instrument 50 is relative to a fixed frame on the robotic arm 40. The pose of the handle controller 38a is then scaled by a scaling function executed by the controller 21a. In aspects, the coordinate position is scaled down and the orientation is scaled up by the scaling function. In addition, the controller 21a also executes a clutching function, which disengages the handle controller 38a from the robotic arm 40. In particular, the controller 21a stops transmitting movement commands from the handle controller 38a to the robotic arm 40 if certain movement limits or other thresholds are exceeded and in essence acts like a virtual clutch mechanism, e.g., limits mechanical input from effecting mechanical output.

The desired pose of the robotic arm 40 is based on the pose of the handle controller 38a and is then passed by an inverse kinematics function executed by the controller 21a. The inverse kinematics function calculates angles for the joints 44a, 44b, 44c of the robotic arm 40 that achieve the scaled and adjusted pose input by the handle controller 38a. The calculated angles are then passed to the robotic arm controller 41c, which includes a joint axis controller having a proportional-derivative (PD) controller, the friction estimator module, the gravity compensator module, and a two-sided saturation block, which is configured to limit the commanded torque of the motors of the joints 44a, 44b, 44c.

With reference to FIG. 5, the surgical robotic system 10 is setup around the surgical table 100. The system 10 includes a plurality of movable carts 60a-d, which may be numbered “1” through “4.” During setup, each of the carts 60a-d are positioned around the surgical table 100. Position and orientation of the carts 60a-d depends on a plurality of factors, such as placement of a plurality of ports 55a-d, which in turn, depends on the surgery being performed. Once the port placement is determined, the ports 55a-d are inserted into the patient, each of the robotic arms 40a-d are aligned to achieve a desired configuration of each of their respective joints, and carts 60a-d are positioned to insert instruments 50 and the endoscopic camera 51 into corresponding ports 55a-d.

With reference to FIGS. 2, 3, and 6, the robotic arm 40 and the setup arm 62 of any the robotic arms 40a-b and the movable carts 60a-d may be configured to achieve an aligned configuration. Aligned configuration denotes any configuration of the robotic arm 40 and the setup arm 62 suitable for operation, storage, transport, etc. The alignment process involves placing each of the joints 44a-c, 46b of the robotic arm 40 and the rotatable base 64 and the joints 63a and 63b of the setup arm 62 (hereinafter “joints” collectively) and adjusting their rotation to achieve the aligned configuration. In particular, rotation of the joints aligns the links 42a-c of the robotic arm 40 and the links 62a-c of the setup arm 62 (hereinafter “links” collectively) into the aligned configuration.

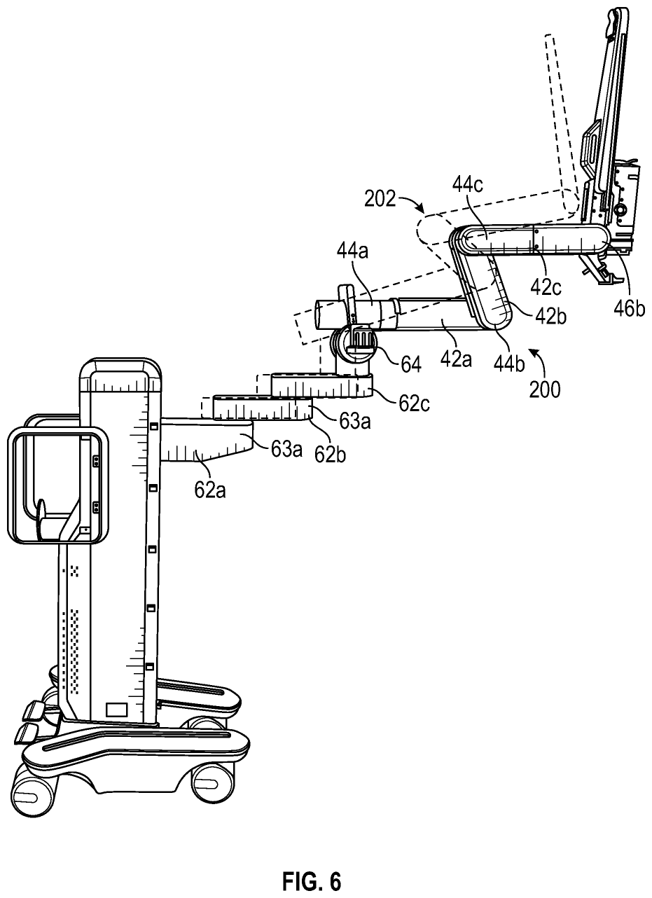

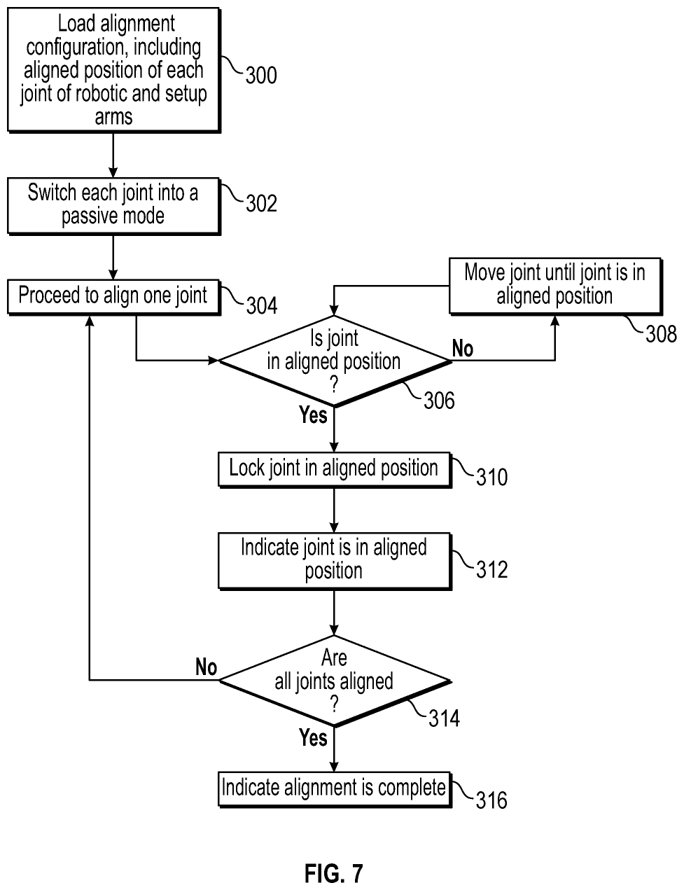

In FIG. 6 the robotic arm 40 and the setup arm 62 are shown in an aligned configuration 200 and a dashed line represents an unaligned configuration 202. The alignment process is shown in FIG. 7 and includes entering the robotic arm 40 and/or the setup arm 62 into alignment mode, which may be done via the controls 65 (FIG. 3) on the robotic arm 40. The alignment process may include an algorithm embodied as software instructions executed by the main cart controller 41a or any other controller of the surgical robotic system 10.

In embodiments, the controls 65 may include a display configured to provide instructions to the user how to align the joints. The aligned configuration may be provided or loaded to the main cart controller 41a of the movable cart 60 by the controller 21a of the control tower 20 at step 300. The aligned configuration may be any configuration for the robotic arm 40 and the setup arm 62, such as a storage configuration, a transport configuration, a surgery or operation configuration, and the like. The surgery configurations may be determined during surgical planning and may include one or more configuration for the robotic arm 40 and/or the setup arm 62 to achieve during operation of the surgical robotic system 10. In further embodiments, the surgery configurations may be adjusted or generated during operation to provide for flexible deployment of the robotic arm 40 and/or the setup arm 62.

The aligned configuration includes a desired position for each of the joints. Once the alignment configuration is loaded, each of the joints is placed into a passive mode at step 302. In this mode each of the joints is freely movable by a user. The brakes of the joints are not engaged, allowing for free movement of each of the joints in response to manual manipulation (e.g., push, pull, raise, lower, etc.) of the links.

At step 304, one of the joints is aligned. Alignment of each joint may proceed in any order or sequence and may be aided by prompts through a user interface of the controls 65. In embodiments, the joints may be aligned from the base joint, namely, the joint 63a since this joint is the first joint coupled to the movable cart 60 and thereafter proceed in the order of closest to furthest joint, namely, joint 63b, the rotatable base 64, the joints 44a-c, etc. In other embodiments, the alignment may occur from the furthest to closest joint, in reverse order described above. More specifically, the joint 46b may be aligned first, follows by the joint 44c, then joint 44b, etc. In additional embodiments, any joint may be aligned in any desired sequence since each position of each of the joints is loaded into the main cart controller 41a.

Whether each of the joints is aligned is determined at step 306. Alignment of each joint may be determined based on position of an actuator of the joint. The position of the actuator may be determined based on rotation of each actuator or any other travel indication. Thus, each joint and/or the links coupled thereto are moved until the joint reaches a position corresponding to an aligned position at step 308. Once the main cart controller 41a determines that the joint has reached the aligned position, the main cart controller 41a locks the joint in the aligned position at step 310. Locking may occur by engaging a brake of the joint and/or disabling the passive mode of the joint and engaging the actuator and preventing any manual movement of the joint and/or the links. The main cart controller 41a may output an audio and/or visual indication on the controls 65 and/or any of the displays 23, 32, 34 that the joint is properly aligned at step 312.

Once one joint is in the aligned position, the main cart controller 41a confirms whether there are any other joints that need to be aligned at step 314 and proceeds to the next joint and repeats the alignment process for each joint until all of the joints have been aligned. At this point, the main cart controller 41a may output an audio and/or visual indication on the controls 65 and/or any of the displays 23, 32, 34 that the entire robotic arm 40 and the setup arm 62 are aligned at step 316.

It will be understood that various modifications may be made to the aspects disclosed herein. In aspects, the color-coded relative position, angular orientation, and operational status of each robotic arm may also be simultaneously viewable on multiple displays. Therefore, the above description should not be construed as limiting, but merely as exemplifications of various aspects. Those skilled in the art will envision other modifications within the scope and spirit of the claims appended thereto.

What is claimed is:

1. A surgical robotic system comprising:
a robotic arm including a plurality of robotic arm joints; and
a controller configured to:
receive an alignment for the robotic arm including an aligned position for each robotic arm joint of the plurality of robotic arm joints;
switch each robotic arm joint of the plurality of robotic arm joints into a passive mode during which each robotic arm joint is manually movable;
track a position of each robotic arm joint of the plurality of robotic arm joints while each robotic arm joint is manually moved into alignment; and
lock in a predetermined sequence each robotic arm joint of the plurality of robotic arm joints once the aligned position for each robotic arm joint is achieved.

2. The surgical robotic system according to claim 1, further comprising:
a display configured to output an indication that each robotic arm joint of the plurality of robotic arm joints is moved into alignment.

3. The surgical robotic system according to claim 2, wherein the display is further configured to output an indication that the robotic arm is in an aligned configuration.

4. The surgical robotic system according to claim 2, wherein the display is configured to output instructions for aligning the robotic arm.

5. The surgical robotic system according to claim 4, wherein instructions include aligning the plurality of robotic arm joints in the predetermined sequence.

6. The surgical robotic system according to claim 1, wherein an alignment corresponds to a configuration of the robotic arm selected from the group consisting of a storage configuration, transport configuration, and an operation configuration.

7. The surgical robotic system according to claim 1, further comprising:
an instrument configured to mechanically engage the robotic arm.

8. The surgical robotic system according to claim 1, further comprising:
a movable cart including a setup arm configured to engage the robotic arm, the setup arm including a plurality of setup arm joints.

9. The surgical robotic system according to claim 8, wherein the controller is further configured to:
receive an alignment for the setup arm including an aligned position for each setup arm joint of the plurality of setup arm joints;
switch each setup arm joint of the plurality of setup arm joints into a passive mode during which each setup arm joint is manually movable;
track a position of each setup arm joint of the plurality of setup arm joints while each setup arm joint is manually moved into alignment; and
lock each setup arm joint of the plurality of setup arm joints once the aligned position for each setup arm joint is achieved.

10. The surgical robotic system according to claim 9, wherein the plurality of robotic arm joints and the plurality of setup arm joints are aligned together.

11. A method for controlling a surgical robotic system, the method comprising:
Receiving an alignment for a robotic arm including a plurality of robotic arm joints, the alignment includes an aligned position for each robotic arm joint of the plurality of robotic arm joints;
switching each robotic arm joint of the plurality of robotic arm joints into a passive mode;
tracking a position of each robotic arm joint of the plurality of robotic arm joints while each robotic arm joint is moved into alignment;
moving each robotic arm joint of the plurality of robotic arm joints while in the passive mode; and
locking each robotic arm joint of the plurality of robotic arm joints once the aligned position for each robotic arm joint is achieved.

12. The method according to claim 11, further comprising:
outputting on a display an indication that each robotic arm joint of the plurality of robotic arm joints is moved into alignment.

13. The method according to claim 11, further comprising:
outputting on a display an indication that the robotic arm is in an aligned configuration.

14. The method according to claim 11, further comprising:
outputting, on a display, instructions for aligning the robotic arm.

15. The method according to claim 14, wherein instructions include aligning the plurality of robotic arm joints in a predetermined sequence.

16. The method according to claim 11, wherein an alignment corresponds to a configuration of the robotic arm selected from the group consisting of a storage configuration, transport configuration, and an operation configuration.

17. The method according to claim 11, further comprising:
mechanically engaging an instrument to the robotic arm.

18. The method according to claim 11, further comprising:
coupling the robotic arm to a movable cart including a setup arm configured to engage the robotic arm, the setup arm including a plurality of setup arm joints.

19. The method according to claim 18, wherein the method further includes:
loading an alignment for the setup arm including an aligned position for each setup arm joint of the plurality of setup arm joints;
switching each setup arm joint of the plurality of setup arm joints into a passive mode during which each setup arm joint is manually movable;
tracking a position of each setup arm joint of the plurality of setup arm joints while each setup arm joint is manually moved into alignment; and
locking each setup arm joint of the plurality of setup arm joints once the aligned position for each setup arm joint is achieved.

20. The method according to claim 19, further comprising:
aligning the plurality of robotic arm joints and the plurality of setup arm joints together.
